(19) United States
(12) Patent Application Publication
(10) Pub. No.: US 2001/0051642 A1
(43) Pub. Date: Dec. 13, 2001

(54) Method for treating Alzheimer's disease

(76) Inventors: Kyunghye Ahn, Ann Arbor, MI (US); Mark Richard Emmerling, Chelsea, MI (US); Taraneh Haske, Ann Arbor, MI (US); Donald J. Hupe, Ann Arbor, MI (US); Judith Sebolt-Leopold, Ann Arbor, MI (US); Harry LeVine III, Ann Arbor, MI (US); Jeffrey David Scholten, Pinckney, MI (US)

Correspondence Address: Charles W. Ashbrook, Warner-Lambert Company, 2800 Plymouth Road, Ann Arbor, MI, 48105, US

(21) Appl. No.: 09/771,529

(22) Filed: Jan. 29, 2001

Related U.S. Application Data
(60) Provisional application No. 60/197,484, filed on Apr. 17, 2000.

(51) Int. Cl. A61K 31/4164; A61K 31/4439; A61K 31/4709

(52) U.S. Cl. 514/341; 514/314; 514/400

(57) ABSTRACT

The present invention provides methods and compositions for inhibiting Aβ synthesis and for treating Alzheimer's disease by administering a farnesyl transferase inhibitor of the formula:

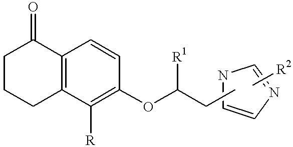

wherein R is hydrogen, alkyl, and substituted alkyl; R¹ is hydrogen, phenyl, or substituted phenyl; and R² is hydrogen or benzyl.

### FIELD OF THE INVENTION

The present invention is directed towards the treatment of senile dementia and Alzheimer's disease by administering a compound that inhibits enzymes known as farnesyl protein transferases.

### BACKGROUND OF THE INVENTION

Alzheimer's disease is characterized by a progressive dementia. The etiology and pathogenesis of this dementia is not well understood. However, it is known that the symptoms of the disease are associated with amyloid plaque deposits, as well as cerebrovascular amyloid and neurofibrillary tangles in the brain and cerebrovasculature. Although non-Alzheimer's disease subjects may have plaques, the number of plaques in Alzheimer's disease patients' brains are typically 5- to 10-fold greater than in age-matched healthy controls. Increased levels of plaques may result from increased rate of synthesis of the components of the plaques, decreased rate of degradation, or some combination of the two.

The primary protein component of plaques is the 42 amino acid (4.2 kDa) β-amyloid peptide (Aβ), which is derived from a family of larger amyloid peptide precursor (APP) proteins. The process by which amyloidosis occurs is not well understood but involves Aβ which is found in extracellular spaces like cerebrospinal fluid (CSF) of the brain and conditioned media of many cell types.

Although there is significant controversy as to the exact role of Aβ in Alzheimer's disease, it is evident that this peptide, and the amyloid fibrils and tangles it produces, are somehow integral to the pathogenesis of the disease. Methods that center around preventing, dissolving, inhibiting, or otherwise ameliorating the detrimental effects of such plaque formation are thus needed. Several classes of compounds have exhibited marginal utility in treating Alzheimer's disease. For example, acetylcholinesterase inhibitors such as tacrine and donepezil have been used clinically (see U.S. Pat. No. 4,816,456). Muscarinic agonists such as milameline have shown marginal efficacy in clinical trials (see U.S. Pat. No. 5,219,872). More recently, statins such as lovastatin and simvastatin have been reported to be useful in preventing and treating Alzheimer's disease (see U.S. Pat. No. 6,080,778). This invention provides a new method to treat Alzheimer's disease by administering compounds known to inhibit enzymes called farnesyl protein transferases.

### SUMMARY OF THE INVENTION

This invention is a method for treating Alzheimer's disease in a mammal comprising administering an effective amount of a farnesyl protein transferase inhibitor selected from the group consisting of:

(S)6-(2-Imidazol-1-yl-1-phenyl-ethoxy)-5-phenethyl-3,4-dihydro-2H-naphthalen-1-one (Compound No. 1);

6-[2-(1 H-Imidazol-4-yl)-ethoxy]-3,4-dihydro-2H-naphthalen-1-one (Compound 2);

E-(+/−)6-(2-Imidazol-1-yl-1-phenyl-ethoxy)-2-thiophen-2-ylmethylene-3,4-dihydro-2H-naphthalen-1-one (Compound 3);

6-[1-(4-Chloro-phenyl)-2-imidazol-1-yl-ethoxy]-3,4-dihydro-2H-napththalen-1-one racemic (Compound 4);

(R)6-(2-Imidazol-1-yl-1-phenyl-ethoxy)-3,4-dihydro-2H-naphthalen-1-one (Compound 5);

6-(2-Imidazol-1-yl-1-phenyl-ethoxy)-4-phenyl-3,4-dihydro-2H-naphthalen-1-one racemic (Compound 6);

6-(2-Imidazol-1-yl-1-phenyl-ethoxy)-5-isopropoxymethyl-3,4-dihydro-2H-naphthalen-1-one (Compound 7);

(S)6-(2-Imidazol-1-yl-1-phenyl-ethoxy)-5-phenylaminomethyl-3,4-dihydro-2H-naphthalen-1-one (Compound 8);

(S)6-(2-Imidazol-1-yl-1-phenyl-ethoxy)-5-[2-(4-fluorophenyl)ethyl]-3,4-dihydro-2H-naphthalen-1-one (Compound 9);

(S)5-Benzenesulfonylmethyl-6-(2-imidazol-1-yl-1-phenyl-ethoxy)-3,4-dihydro-2H-naphthalen-1-one (Compound 10);

(S)6-(2-Imidazol-1-yl-1-phenyl-ethylsulfanyl)-5-phenethyl-3,4-dihydro-2H-naphthalen-1-one (Compound 11);

(S)6-(2-Imidazol-1-yl-1-phenyl-ethoxy)-5-(2-pyridin-2-yl-ethyl)-3,4-dihydro-2H-naphthalen-1-one (Compound 12);

6-(2-Imidazol-1-yl-1-phenyl-ethoxy)-5-(2-pyridin-4-yl-ethyl)-3,4-dihydro-2H-naphthalen-1-one (Compound 13);

4-(5-Oxo-1-phenethyl-5,6,7,8-tetrahydro-naphthalen-2-yloxy)-4-phenyl-butyric acid (Compound 14);

6-[2-(3-Benzyl-3H-imidazol-4-yl)-ethoxy]-5-phenethyl-3,4-dihydro-2H-naphthalen-1-one; trifluoro-acetate (Compound 15);

(S)[1-{(4-Benzyloxy-benzyl)-[(2-methyl-2-phenyl-propylcarbamoyl)-methyl]-carbamoyl}-2-(3H-imidazol-4-yl)-ethyl]-carbamic acid benzyl ester (Compound 16);

(S)[2-(1H-Imidazol-4-yl)-1-((4-methyl-benzyl)-{[(1-phenyl-cyclobutylmethyl)-carbamoyl]-methyl}-carbamoyl)-ethyl]-carbamic acid benzyl ester (Compound 17);

1-Methyl-4-(3-chlorophenyl)-6-[(4-chlorophenyl)-(1-methylimidazol-5-yl)aminomethyl]-2,3-dihydroquinolin-2-one (Compound 18); and

(S)[1-{(4-Benzyloxy-benzyl)-[(2-benzyloxy-ethylcarbamoyl)-methyl]-carbamoyl}-2-(1H-imidazol-4-yl)ethyl]-carbamic acid benzyl ester (Compound 19).

In a preferred embodiment, Compound No. 1 is administered to patients suffering from Alzheimer's disease and in need of treatment.

A further embodiment is a method for inhibiting amyloidosis in a patient comprising administering an amyloidosis inhibiting amount of one of the above compounds.

Still another embodiment is a method for inhibiting amyloid plaque formation in a cell population comprising contacting the cell population with an effective amount of one of the above compounds.

### BRIEF DESCRIPTION OF FIGURES

FIG. 1: CHO cells transfected with Swedish mutation Amyloid Precursor protein (CHO-APPsw) were incubated with various HMG-CoA reductase inhibitors in serum free media overnight at 37° C. Secreted Aβ in the media was quantified by sandwich ELISA. All HMG-CoA reductase inhibitors reduced Aβ secretion from these cells in a dose-dependent manner.

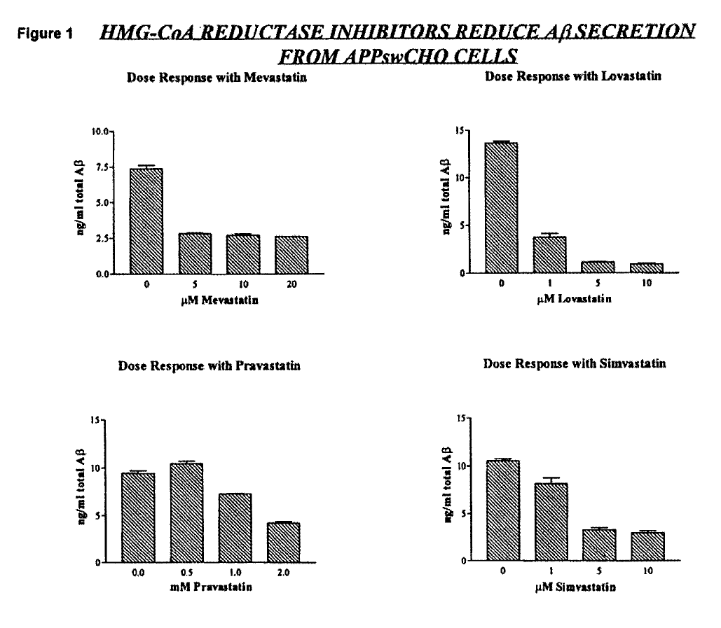

FIG. 2: CHO cells transfected with Swedish mutation Amyloid Precursor protein (CHO-APPsw) were incubated with various farnesyl protein transferase inhibitors in serum free media overnight at 37° C. Secreted Aβ in the media was quantified by sandwich ELISA. Invention compounds reduce Aβ secretion in a dose-dependent manner.

### DETAILED DESCRIPTION OF THE INVENTION

The farnesyl protein transferase inhibitors to be administered to patients according to this invention are known compounds. The preferred compounds are dihydro naphthalenones having the following formula:

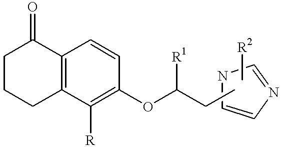

where R, R¹, and R² are shown below in Table 1 for specific compounds.

TABLE 1

| Com- | | | R² |
|---|---|---|---|
| pound | | | (point of attachment |
| No. | R | R¹ | of imidazole ring) |
| 1 | —CH₂CH₂Ph | Ph-(S-isomer) | H-(imidazol-1-yl) |
| 2 | H | H | H-(imidazol-4-yl) |
| 4 | H | 4-ClPh | H-(imidazol-1-yl) |
| 5 | H | Ph-(R-isomer) | H-(imidazol-1-yl) |
| 7 | —CH₂0-iPr | Ph-(S-isomer) | H-(imidazol-1-yl) |
| 8 | —CH₂NHPh | Ph-(S-isomer) | H-(imidazol-1-yl) |
| 9 | —CH₂CH₂-4-F—Ph | Ph-(S-isomer) | H-(imidazol-1-yl) |
| 10 | —CH₂SO₂Ph | Ph-(S-isomer) | H-(imidazol-1-yl) |
| 12 | —CH₂CH₂-2-pyridyl | Ph-(S-isomer) | H-(imidazol-1-yl) |
| 13 | —CH₂CH₂-4-pyridyl | Ph-(S-isomer) | H-(imidazol-1-yl) |
| 15 | —CH₂CH₂Ph | H | 3-CH₂Ph-(imidazol- |
| | | | 4-yl) trifluoroacetate |

Other compounds that are especially preferred for use according to this invention include:

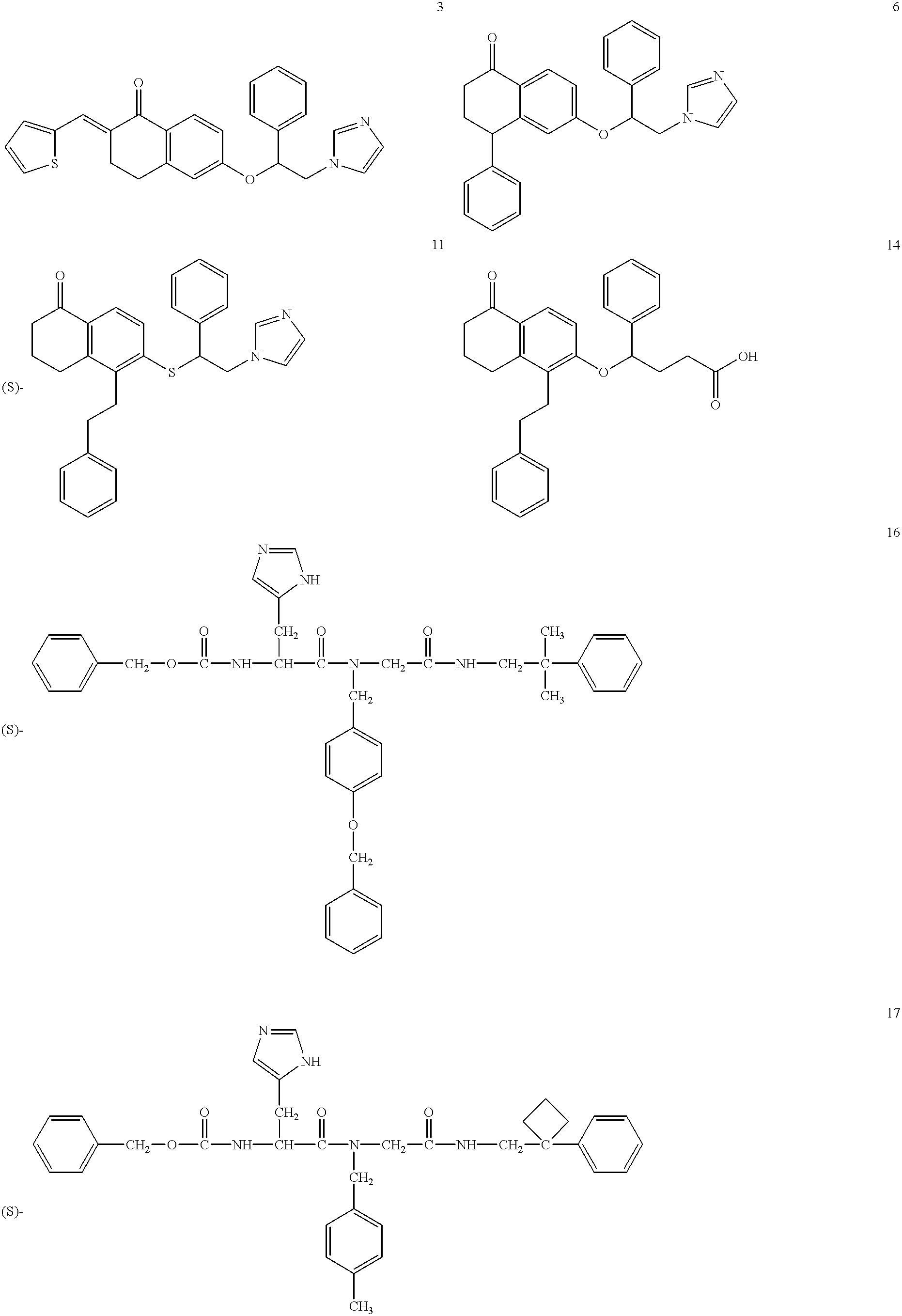

As used herein, the terms “mammal” and “patient” means animals such as humans, dogs, cats, and horses.

The term “effective amount” means that quantity of one of the compounds described herein required to inhibit amyloid plaque formation, or to otherwise treat the symptoms of Alzheimer's disease.

The present invention provides for the administration of pharmaceutical compositions comprising one or more of the above compounds in order to ameliorate the effects of amyloidosis in for example, Alzheimer's disease, Down syndrome, and other disorders characterized by the deposition of amyloid plaques. The pharmaceutical compositions of the invention can take any of a wide variety of oral and parenteral dosage forms. The dosage forms comprise as the active component a farnesyl transferase inhibitor.

For preparing pharmaceutical compositions, one uses inert, pharmaceutically acceptable carriers that can be either solid or liquid. Solid form preparations include powders, tablets, dispersible granules, capsules, cachets, and suppositories. A solid carrier can be one or more substances which may also act as diluents, flavoring agents, solubilizers, lubricants, suspending agents, binders, or tablet disintegrating agents; it can also be an encapsulating material. In powders, the carrier is a finely divided solid which is in admixture with the finely divided active compounds. In the tablet, the active compounds are mixed with carrier having the necessary binding properties in suitable proportions and compacted in the shape and size desired. The powders and tablets preferably contain from 5% or 10% to about 70% of active ingredients.

Suitable solid carriers are magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like. The term “preparation” is intended to include the formulation of the active compounds with encapsulating materials as carrier, providing a capsule in which the active components (with or without other carriers) are surrounded by carrier, which are thus in association with it. Similarly, cachets are included. Tablets, powders, cachets, and capsules can be used as solid dosage forms suitable for oral administration.

Liquid form preparations include solutions, suspensions, and emulsions. As an example may be mentioned water or water-propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active components in water with viscous material, i.e., natural or synthetic gums, resins, methyl cellulose, sodium carboxymethyl cellulose, and other well-known suspending agents.

Preferably, the pharmaceutical preparation is in unit dosage form. The quantity of the active compound to be used in a unit dose of preparation may be varied or adjusted from about 0.01 to 500 mg/kg body weight, preferably 0.03 mg/kg body weight to less than 100 mg/kg body weight of the active ingredient, according to the particular application and the potency of the active ingredients. In those embodiments where there is more than one isoprenylation inhibitor or there is an isoprenylation inhibitor being used in combination with a second anti-Alzheimer's disease agent, the preparation may be subdivided into unit doses containing appropriate quantities of the two agents individually or as a combination, i.e., in a mixture. The unit dosage form can be a packaged preparation, the package containing discrete quantities of preparation, for example, picketed tablets, capsules, and powders in vials or ampoules. The unit dosage form can also be a capsule, cachet, or tablet itself or it can be the appropriate number of any of these in packaged form. Additionally, the unit dosage form may be a dividable form having one agent in one part and the other agent in the other part, such as, a dividable capsule, a dividable package, or a two-part ampoule, vial, or the like.

The pharmaceutical compositions preferably are constituted so that they can be administered parenterally or orally. Solutions of the active compounds as free bases and free acids or pharmaceutically acceptable salts can be prepared in water suitable mixed with a surfactant such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants. The prevention of the action of microorganisms can be brought about by various antibacterial and antifungal agents, for example, paragens, chlorobutanol, phenol, sorbic acid, thimerosal, and the like. In many cases, it will be preferable to include isotonic agents, for example, sugars or sodium chloride. Prolonged absorption of the injectable compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients, into a sterile vehicle which contains the basic dispersion medium and the required other ingredients from those enumerated above. In the case of the sterile powders for the preparation of sterile injectable solutions, the preferred methods of preparation are vacuum drying and the freeze-drying technique which yield a powder of active ingredients plus an additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active ingredient, its use in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suitable as unitary dosages for the mammalian subjects to be treated; each unit containing a predetermined quantity of active materials calculated to produce the desired therapeutic effect in association with the required pharmaceutical carrier. The specification for the novel dosage unit forms of the invention are dictated by and directly dependent on (a) the unique characteristics of the active materials and the particular therapeutic effect to be achieved, and (b) the limitation inherent in the art of compounding such active materials for the treatment of disease in living subjects having a diseased condition in which bodily health is impaired as herein disclosed in detail.

The principal active ingredients are compounded for convenient and effective administration in effective amounts with a suitable pharmaceutically acceptable carrier in dosage unit form as disclosed herein. A unit parenteral dosage form can, for example, contain the principal active compound, i.e., Compound No. 1, in amounts ranging from about 0.5 to about 1000 mg, with from about 2 to 250 mg being preferred. The daily parenteral doses for mammalian subjects to be treated ranges from about 0.01 to about 100 mg/kg body weight of the farnesyl transferase. The preferred daily dosage range is from about 0.1 to about 10.0 mg/kg body weight.

### EXAMPLES

The following examples demonstrate the synthesis and biological evaluation of compounds to be used in the invention. It should be appreciated by those of skill in the art that the techniques disclosed in the examples represent techniques discovered by the inventors to function well in the practice of the invention, and thus can be considered to constitute a preferred mode for its practice. However, those of skill in the art should, in light of the present disclosure, appreciate that many changes can be made in the specific embodiment disclosed and still obtain a like or similar result without departing from the spirit and scope of the invention. All references cited herein are incorporated by reference.

### Example 1

### Synthesis of Typical Farnesyl Protein Transferase Inhibitors

The compounds to be used in the present method are generally well-known, and can be prepared by any of several standard synthetic processes. Many of the compounds (e.g., Compound 2) are described in U.S. Pat. No. 6,133,303. Scheme 1 shows a typical synthesis of a hydroxyalkyl-imidazole intermediate, starting from a readily available cyanoalkyl-imidazole. The secondary nitrogen of the imidazole is protected, for instance, with a t-BOC group, and the desired R³ group (such as benzyl for instance) is then added by reacting the protected imidazole with an R³-L, where L is a leaving group such as halo or hydroxy. The reaction generally is carried out in the presence of an acid such as sulfuric acid, which also removes the N-protecting group. The cyano group is next hydrolyzed to a carboxylic acid by reaction with a strong base, and the acid can be esterified by reaction with an alcohol. The ester is then readily reduced by reaction with lithium aluminum hydride or similar reducing agent to give the desired hydroxyalkyl-imidazole. The hydroxyalkyl-imidazole is then readily reacted with a suitably substituted 3,4-dihydro-2H-naphthalen-1-one, for example in the presence of a dehydrating agent, to produce the compound to be used in the method of this invention.

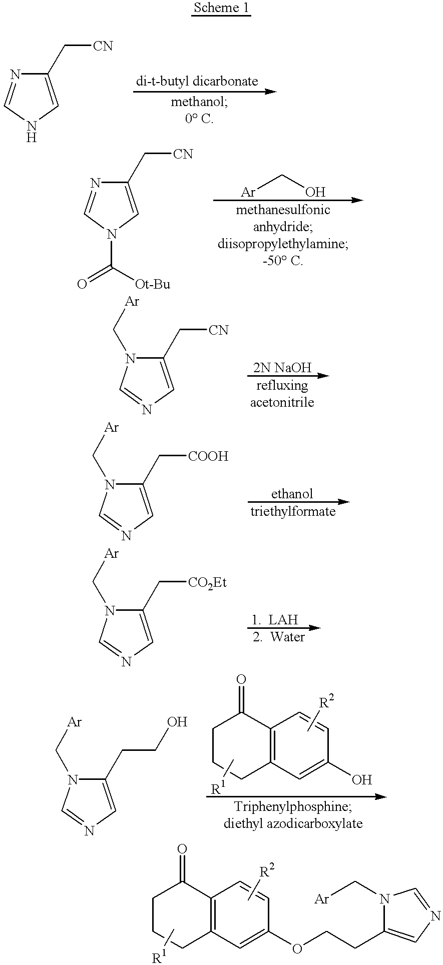

### Example 1a

### Synthesis of 6-{2-[3-(4-methoxy-3-methyl-benzyl)-3H-imidazol-4-yl]-ethoxy}-3,4-dihydro-2H-naphthalen-1-one

1. 4-Cyanomethyl-imidazole-l-carboxylic acid tert-butyl ester

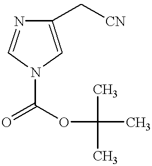

A solution of 4(5)-cyanomethylimidazole (12.2 g, 0.114 mol) in methanol (150 mL) is cooled to 0° C., and a solution of di-tert-butyl dicarbonate (30 g, 0.137 mol) in methanol (75 mL) is then added. The reaction is stirred under a nitrogen atmosphere and is left to warm to room temperature overnight. The solution is concentrated in vacuo and the residue taken up in isopropyl ether and concentrated in vacuo. The residue is taken up in isopropyl ether and chilled for several hours. The crystalline product is collected by filtration. The mother liquor is purified by flash chromatography (chloroform:acetone/9:1) to give 5.7 grams of the title compound (27% yield).

2. [3-(4-Methoxy-3-methyl-benzyl)-3H-imidazol-4-yl]-acetonitrile

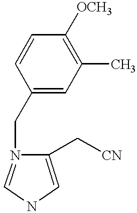

A solution of methanesulfonic anhydride (7.67 g, 0.044 mol) in methylene chloride is cooled to −50° C. Dropwise, a solution of 4-methoxy-3-methyl-benzyl alcohol (6.69 g, 0.044 mol), diisopropylethylamine (7.67 mL, 0.044 mol), and methylene chloride (60 mL) is added. The solution is stirred at −50° C. for 15 minutes and warmed to −20° C. over 15 minutes. The reaction vessel is cooled back to −50° C. and a solution of the product from 1,4-cyanomethyl-imidazole-1-carboxylic acid tert-butyl ester (9.07 g, 0.0438 mol), in methylene chloride (60 mL) is added dropwise. The reaction is warmed to room temperature and stirred overnight. A solution 0.25 M potassium phosphate buffer (pH 7, 300 mL) is added to the reaction mixture and vigorously stirred for 30 minutes. The organic phase is separated, washed with the phosphate buffer, dried over magnesium sulfate, and concentrated in vacuo. The product is purified by flash chromatography (0-1% methanol in chloroform) to give an oil which is dissolved in methylene chloride; the solution is evaporated and dried at 0.5 mm, to give a crystallized product (4. 3 g, 41% yield).

3. 3-(4-Methoxy-3-methyl-benzyl)-3H-imidazol-4-yl]-acetic acid

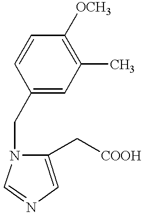

The product obtained in 2,[3-(4-methoxy-3-methyl-benzyl)-3H-imidazol-4-yl]-acetonitrile (4.3 g, 0.0 178 mol), is suspended in 2N NaOH (18 mL), and heated to reflux for 4 hours. The solution is cooled and neutralized with 1 N HCl (36 mL), diluted with ethanol (100 mL) and concentrated in vacuo. The residue is taken up in ethanol (250 mL), the precipitate filtered and the solution filtered and concentrated in vacuo. The residue is triturated with hot ethyl acetate, cooled and filtered to give an off-white solid (4.5 g, 97% yield); mp: 117-121° C.

4. [3-(4-Methoxy-3-methyl-benzyl)-3H-imidazol-4-yl]-acetic acid ethyl ester

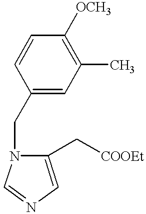

The product obtained in 3, [3-(4-methoxy-3-methyl-benzyl)-3H-imidazol-4-yl]-acetic acid. (3.4 g, 0.013 mol) is dissolved in ethanol (100 mL) and triethylorthofornate (5 mL). This solution is saturated with dry HCl and the reaction is heated to reflux for 5 hours. The solution is concentrated in vacuo; the residue is triturated with ethyl acetate and dried overnight at 65° C., in vacuo to give 3.95 g (93% yield) of the desired product.

5. 2-[3-(4-Methoxy-3-methyl-benzyl)-3H-imidazol-4-yl]-ethanol

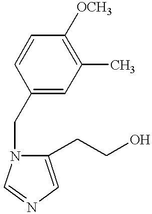

A suspension of the product from 4, [3-(4-Methoxy-3-methyl-benzyl)-3H-imidazol-4-yl]-acetic acid ethyl ester (3.9 g, 0.012 mol), in tetrahydrofuran (250 mL) is stirred vigorously under a nitrogen atmosphere for 30 minutes. Lithium aluminium hydride (0.5 g, 0.013 mol) is then added slowly in 5 portions. The reaction is stirred 30 minutes at 0° C., warmed to room temperature for 3 hours, and then refluxed for 1 hour. The reaction is quenched by the dropwise addition of water (0.9 mL) and the Li/Al salts are removed by filtration through Celite, rinsing thoroughly with tetrahydrofuran:methanol (95:5). The filtrate is concentrated in vacuo to give the product (2.37 g, 80% yield).

6. 6-{2-[3-(4-Methoxy-3-methyl-benzyl)-3H-imidazol-4-yl]-ethoxy}-3,4-dihydro-2H-naphthalen-1-one

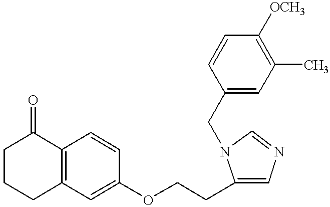

### Compound 20

6-Hydroxy-tetralone (0.48 g, 0.003 mol) is dissolved in tetrahydrofuran (30 mL). Triphenylphosphine (1.11 g, 0.0042 mol) is then added followed by the product from 5, 2-[3-(4-methoxy-3-methyl-benzyl)-3H-imidazol-4-yl]-ethanol (0.75 g, 0.003 mol). A solution of diethyl azodicarboxylate (0.6 mL, 0.0038 mol) in tetrahydrofuran (10 mL) is added slowly under a nitrogen atmosphere. The reaction is stirred at room temperature overnight under a nitrogen atmosphere. The precipitate is filtered, and the filtrate concentrated in vacuo. The residue is taken up in ethyl acetate, washed three times with brine, dried over magnesium sulfate, filtered, and concentrated in vacuo. The product is purified by flash chromatography (5% methanol in chloroform) to give 0.51 g (43% yield) material which is further purified by reverse phase HPLC (C-18 column; 22×250 mm; 0.1 mm; 300 Å; gradient: 10% to 40% acetonitrile; 0.1% trifluoroacetic acid; against 1% aqueous trifluoroacetic acid; 100 minutes; 13 mL/minute), to give 0.070 g of Compound 20 (6% yield).

MS: APCI: M+1: 391.2 (M: 390.5).

Analysis calculated for C₂₄H₂₆N₂O₃.1.25 CF₃COOH: C, 59.72; H, 5.15; N, 5.26.

Found: C, 59.71; H, 5.13; N, 5.19.

### Example 2

### Synthesis of 6-((1S)-2-Imidazolyl-1-phenylethoxy)-5-(2-phenylethyl)-3,4-dihydro-2H-naphthalen-1-one (Compound 1)

1. 6-Methoxy-5-phenylethynyl-3,4-dihydro-2H-napthalen-1-one

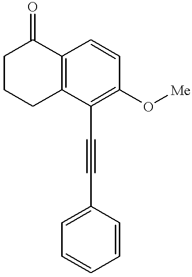

5-Bromo-6-methoxy-3,4-dihydro-2H-napthalen-1-one (10.7 g, 0.04 mol) (Z. Chem. 1970;10:70) and phenylacetylene (8.2 g, 0.08 mol) are added to a mixture of dimethylformamide (80 mL) and triethylamine (40 mL). The mixture is sparged with nitrogen gas, and copper iodide (228 mg) is added. Dichlorobis-(triphenyl-phosphine)palladium(II) (1.12 g) and butylated hydroxytoluene (0.08 g) are added, and the resulting reaction mixture is heated to 108° C. for 2 hours. Another portion of phenylacetylene (16 g, 0.16 mol) is slowly added over 3 hours followed by heating to 108° C. for 18 hours. The mixture is then evaporated in vacuo, and the residue is taken up into ethyl ether. Insoluble material is decanted. The organic phase is washed with 1N citric acid and then brine, dried over anhydrous magnesium sulfate, filtered and evaporated in vacuo to a crude solid (32 g). The material is purified by chromatography on 400 g silica gel, eluted with a mixture of ethyl acetate/hexane (10:90). The product is obtained as a solid (5.89 g, 53% yield). NMR spectrum is consistent with structure. MS: APCI: M+1, 277.2 (M: 276.34).

2. 6-Methoxy-5-phenethyl-3,4-dihydro-2H-napthalen-1-one

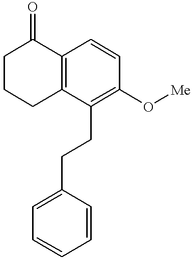

6-Methoxy-5-phenylethynyl-3,4-dihydro-2H-napthalen-1-one (5.69 g, 0.021 mol) and 5% palladium on barium sulfate (1.0 g) are added to tetrahydrofuran (100 mL) followed by pressurization to 49 psi with hydrogen gas. At regular intervals, 3 additional 1 g portions of 5% palladium on barium sulfate are added over 36 hours during additional treatment with hydrogen gas at 49 psi. The mixture is then filtered and evaporated in vacuo to a solid (5.68 g, 98% yield). NMR spectrum is consistent with structure. MS: APCI: M+1, 281.2 (M: 280.37).

3. 6-Hydroxy-5-phenethyl-3,4-dihydro-2H-napthalen-1-one

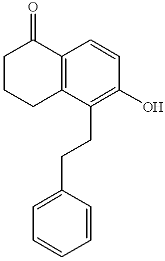

6-Methoxy-5-phenethyl-3,4-dihydro-2H-napthalen-1-one (5.39 g, 0.192 mol) and sodium cyanide (4.71 g, 0.096 mol) are added to dimethylsulfoxide (30 mL) and heated to 180° C. for 18 hours. The mixture is poured, while hot, into water (200 mL), washed with ethyl ether, and acidified to pH 1 with conc. HCl. The mixture is extracted with ethyl ether, washed with brine, dried over anhydrous magnesium sulfate, and filtered. The filtrate is concentrated in vacuo, giving a solid (2.87 g, 56% yield). NMR spectrum is consistent with structure. MS: APCI: M+1, 267.1 (M: 266.3)

4. 6-((1S)-2-Imidazolyl-1-phenylethoxy)-5-(2-phenylethyl)-3,4-dihydro-2H-naphthalen-1-one

To tetrahydrofuran (50 mL) is added 6-hydroxy-5-phenethyl-3,4-dihydro-2H-napthalen-1-one (2.67 g, 10 mmol), (R)-2-imidazol-1-yl-1-phenyl-ethanol (2.15 g, 11.4 mmol), and triphenylphosphine (3.94 g, 15 mmol). A solution of diethylazodicarboxylate (2.61 g, 15 mmol) in tetrahydrofuran (25 mL) is added over 1 hour with cooling to 5° C. After stirring for 18 hours at 25° C., the mixture is evaporated in vacuo, and the residue is suspended in ethyl ether and 1N citric acid, washed exhaustively with ethyl ether and the pH adjusted to 14 with 6N NaOH. The aqueous phase is extracted with ethyl ether, which is subsequently separated, washed with brine, dried over anhydrous magnesium sulfate and filtered. The filtrate is evaporated to a solid (Compound 1) that is purified by recrystallization and obtained as a solid (2.22 g, 51% yield). NMR spectrum is consistent with structure. MS: APCI: M+1, 437.3 (M: 436.6).

Calcd. for C₂₅H₂₈N₂O₂, 0.1 CHCl₃, 0.25 H₂O:

Theory: C. 79.79, H 6.46, N 6.36.

Found: C. 79.47, H 6.52, N 6.42.

The following biological assays establish that the foregoing compounds are inhibitors of farnesyl protein transferase, and that such compounds are useful to inhibit amyloid accumulation, and thus are useful to treat Alzheimer's disease.

### Example 3

### Protein Farnesyl Transferase (PFT) Inhibitory Activity

The PFT (or FPT) inhibitory activity of compounds of the present invention was assayed in HEPES buffer (pH 7.4) containing 5 mM potassium phosphate and 20 μM ZnCl₂. The solution also contained 5 mM DTT (dithiothreitol), 5 mM MgCl₂, and 0.1% PEG 8000. Assays were performed in 96 well plates (Wallec) and employed solutions composed of varying concentrations of a compound of the present invention in 10% DMSO (dimethylsulfoxide). Upon addition of both substrates, radiolabeled farnesyl pyrophosphate ([1³H], specific activity 15-30 Cl/mmol, final concentration 134 μM) and (biotinyl)-Ahe-Thr-Lys-Cys-Val-Ile-Met ([3aS[3a alpha, 4 beta, 6a alpha]-hexahydro-2-oxo-1H-thieno[3,4-d]imidazole-5-pentanoic acid]-[7-aminoheptanoic acid]-Thr-Lys-Cys-Val-Ile-Met) (Ahe is 7-aminoheptanoic acid, Thr is threonine, Lys is lysine, Cys is cysteine, Val is valine, Ile is isoleucine, and Met is methionine) (final concentration 0.2 μM), the enzyme reaction was started by addition of SF9 affinity purified rat FPT. After incubation at 30° C. for 30 minutes, the reaction was terminated by diluting the mixture 2.5-fold with a stop buffer containing 1.5 M magnesium acetate, 0.2 M H₃PO₄, 0.5% BSA (bovine serum albumin), and strepavidin beads (Amersham) at a concentration of 1.3 mg/mL. After allowing the plate to settle for 30 minutes at room temperature, radioactivity was quantitated on a microBeta counter (Model 1450, Wallec). The assay was also carried out without 5 mM potassium phosphate. The IC₅₀ values (the micromolar amount of invention compound required to inhibit enzyme activity by 50%) of representative compounds are shown in Table 2.

TABLE 2
Farnesyl Transferase Inhibition

| | IC₅₀ (μm) | IC₅₀ |
|---|---|---|
| | Hepes + 5 mM | (μm) |
| Compound No. | Phosphate | Hepes |
| 20 | 0.022 | 0.024 |

### Example 4

This is an in vivo assay designed to show the ability of test compounds to inhibit amyloid aggregation. CHO cells transfected with human AβPP bearing the Swedish mutation AβPP(CHO-AβPPsw) were treated with a farnesyl transferase inhibitor (FTI) (e.g., compounds from Table 1). The assay was carried out as follows:

Materials and Methods

Cell Culture

CHO cells transfected with Swedish mutation Amyloid Precursor Protein (CHO-APPsw) were maintained in Dulbecco's Modified Eagle Medium (DMEM) media supplemented with nonessential amino acids (NEAA, Gibco-BRL Company) and 10% (v/v) heat inactivated Fetal Calf Serum (Hyclone Company), penicillin and streptomycin (Gibco-BRL).

Cell Viability Assay

Cytotoxicity of the compounds used in these experiments was determined using Live/Dead Viability Kit (Molecular Probes, Eugene, Oreg., USA). Live cells were recognized by measuring intracellular esterase activity using a green fluorescent polar tracer (calcein AM). Based on these results, appropriate concentrations of the compounds were used.

AβPP Western Blotting

Cells were grown in a 12-well plate to confluency. Cells were washed with serum-free media and incubated with various compounds overnight at 37 degrees. Media was collected, and the cells were washed with phosphate-buffered saline (PBS) and lysed in sodium dodecyl sulfate (SDS) sample buffer (without dithiothreitol (DTT). Protein content determinations were done using BCA Protein Assay (Pierce, Rockford, Ill., USA). Using SDS-PAGE (polyacrylamide gel electrophoresis) and electroblot onto nitrocellulose membrane (Novex, San Diego, Calif., USA) proteins were separated. Immunoblots of the Amyloid Precursor Protein (AβPP) metabolites from media and cell lysate were probed with various antibodies. C-terminal specific anti-serum R57 (Mehta et al., Neurosci. Lett. 1998;241:13-16) was used to detect cellular AβPP and α, β, and γ secretase fragments. The N-terminus specific anti-serum 22C.11 (Boehringer Mannheim Corp., Indianapolis, Ind., USA) was used to recognize cellular and secreted BAPP. The primary antibody was detected with a secondary biotinylated horseradish peroxidase-conjugated antibody (Amersham, Arlington, Ill., USA). Bound antibody was detected with enhanced chemiluminescent detection system (Amersham, Arlington, Ill., USA).

ELISA

Secreted Aβ in the media was quantified by sandwich ELISA as described previously (Carroll et al., Biochem. Biophy. Res. Commun. 1995;210:345-349; Raby et al., Neurochem. 1998;71:2505-2509). The capture antibodies used to coat the microtiter plate were monoclonal antibody 6E10 (Senetek plc, St. Louis, Mo., USA) which binds to residues 1-16 of the Aβ peptide, r163 which reacts only with Aβ40, or r165 which specifically recognizes Aβ42 (Mehta et al., 1998). The polyclonal antibodies were purified on a protein G-Sepharose column prior to use. Biotinylated monoclonal antibody 4G8 which recognizes amino acids 17-24 of Aβ (Senetek plc, St. Louis, Mo., USA) was used as the detection antibody. Alkaline phosphatase-conjugated strepavidin (Zymed Laboratories, San Francisco, Calif., USA) was used to bind the biotinylated detection antibody followed by Attophos (JBL Scientific, St. Luis Obispo, Calif., USA), the substrate for alkaline phosphatase. Fluorescence was measured using a Cytofluor plate reader (Perspective Biosystems, Framingham, Mass., USA) set at 450 nm excitation and 560 emission. Synthetic Aβ40 and Aβ42 peptides used for standard curves were purchased from California Peptide (Napa, Calif., USA).

Table 3 below shows the activity of several invention compounds in the foregoing assay.

TABLE 3
Aβ Secretion Inhibitory Activity

| | Effect on Aβ Secretion (Concentration) (IC₅₀) |
|---|---|
| Compound No. | μM |
| 1 | 0.7 |
| 2 | 7.6 |
| 3 | 4.5 |
| 4 | 25 |
| 5 | 1.5 |
| 6 | 3.1 |
| 7 | 37.9 |
| 8 | 7.6 |
| 9 | 4.5 |
| 10 | 25.4 |
| 11 | 1.5 |
| 12 | 12.0 |
| 13 | 11.0 |
| 14 | 13.8 |
| 15 | 1.5 |
| 16 | 50 |
| 17 | 10 |
| 18 | 6 |
| 19 | 10 |

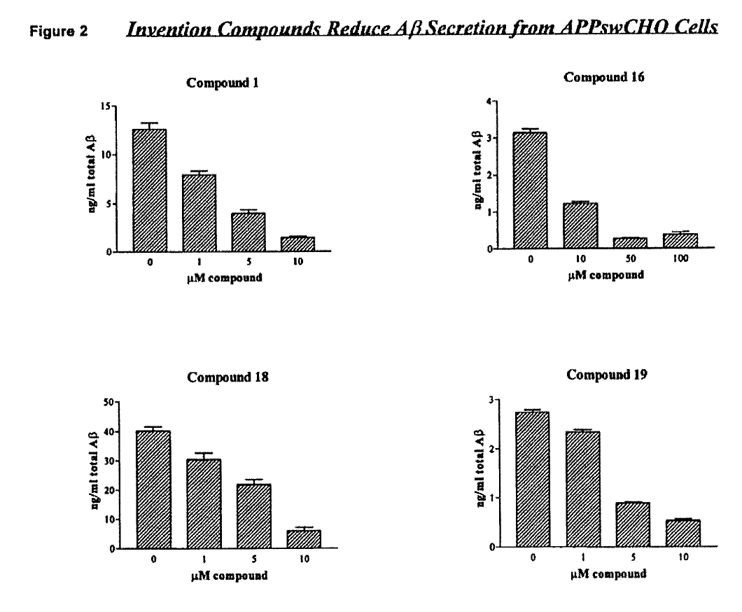

The foregoing assay establishes that the farnesyl transferase inhibitors described above are potent inhibitors of amyload formation, and are thus useful to treat Alzheimer's disease. For example, treatment of the transfected CHO cells with 10 μM compound No. 1 resulted in a 6.5-fold decrease in secreted Aβ. The dose-dependent effect on Aβ secretion of several invention compounds is shown graphically in FIG. 2.

The inhibitory activity of the invention compounds, as established in the foregoing assays, demonstrates that the compounds are useful in preventing amyloid aggregation, and are thus useful to treat Alzheimer's disease. The compounds will be used in the form of pharmaceutical formulations, and the following examples illustrate typical dosage forms.

### Example 5

Tablet Formulation

| Ingredient | Amount |
|---|---|
| Compound No. 1 | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

Compound No. 1 is mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for prevention and treatment of Alzheimer's disease.

### Example 6

Parenteral Solution

In a solution of 700 mL of propylene glycol and 200 mL of water for injection is added 20.0 g of Compound No. 8. The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of Compound No. 8), and sealed under nitrogen. The solution is administered by injection to a patient suffering from Alzheimer's disease and in need of treatment.

The invention and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A method of inhibiting amyloid plaque formation in a cell population comprising contacting said cell population with an effective amount of a compound selected from:
(S) 6-(2-Imidazol-1-yl-1-phenyl-ethoxy)-5-phenethyl-3,4-dihydro-2H-naphthalen-1-one;
6-[2-(1H-Imidazol-4-yl)-ethoxy]-3,4-dihydro-2H-naphthalen-1-one;
E-(+/−)6-(2-Imidazol-1-yl-1-phenyl-ethoxy)-2-thiophen-2-ylmethylene-3,4-dihydro-2H-naphthalen-1-one;
6-[1-(4-Chloro-phenyl)-2-imidazol-1-yl-ethoxy]-3,4-dihydro-2H-napththalen-1-one racemic;
(R)6-(2-Imidazol-1-yl-1-phenyl-ethoxy)-3,4-dihydro-2H-naphthalen-1-one;
6-(2-Imidazol-1-yl-1-phenyl-ethoxy)-4-phenyl-3,4-dihydro-2H-naphthalen-1-one racemic;
6-(2-Imidazol-1-yl-1-phenyl-ethoxy)-5-isopropoxymethyl-3,4-dihydro-2H-naphthalen-1-one;
(S) 6-(2-Imidazol-1-yl-1-phenyl-ethoxy)-5-phenylaminomethyl-3,4-dihydro-2H-naphthalen-1-one;
(S)6-(2-Imidazol-1-yl-1-phenyl-ethoxy)-5-[2-(4-fluorophenyl)ethyl]-3,4-dihydro-2H-naphthalen-1-one;
(S)5-Benzenesulfonylmethyl-6-(2-imidazol-1-yl-1-phenyl-ethoxy)-3,4-dihydro-2H-naphthalen-1-one;
(S)6-(2-Imidazol-1-yl-1-phenyl-ethylsulfanyl)-5-phenethyl-3,4-dihydro-2H-naphthalen-1-one;
(S)6-(2-Imidazol-1-yl-1-phenyl-ethoxy)-5-(2-pyridin-2-yl-ethyl)-3,4-dihydro-2H-naphthalen-1-one;
6-(2-Imidazol-1-yl-1-phenyl-ethoxy)-5-(2-pyridin-4-yl-ethyl)-3,4-dihydro-2H-naphthalen-1-one;
4-(5-Oxo-1-phenethyl-5,6,7,8-tetrahydro-naphthalen-2-yloxy)-4-phenyl-butyric acid;
6-[2-(3-Benzyl-3H-imidazol-4-yl)-ethoxy]-5-phenethyl-3,4-dihydro-2H-naphthalen-1-one; trifluoro-acetate;
(S)[1-{(4-Benzyloxy-benzyl)-[(2-methyl-2-phenyl-propylcarbamoyl)-methyl]-carbamoyl}-2-(3H-imidazol-4-yl)-ethyl]-carbamic acid benzyl ester;
(S)[2-(1H-Imidazol-4-yl)-1-((4-methyl-benzyl)-{[(1-phenyl-cyclobutylmethyl)-carbamoyl]-methyl}-carbamoyl)-ethyl]-carbamic acid benzyl ester;
1-Methyl-4-(3-chlorophenyl)-6-[(4-chlorophenyl)-(1-methylimidazol-5-yl)aminomethyl]-2,3-dihydroquinolin-2-one; and
(S)[1-{(4-Benzyloxy-benzyl)-[(2-benzyloxy-ethylcarbamoyl)-methyl]-carbamoyl}-2-(1H-imidazol-4-yl)ethyl]-carbamic acid benzyl ester.

2. The method of claim 1, wherein the compound administered is
(S) 6-(2-Imidazol-1-yl-1-phenyl-ethoxy)-5-phenethyl-3,4-dihydro-2H-naphthalen-1-one.

3. The method of claim 1, wherein said cell is in culture.

4. The method of claim 1, wherein said cell population is in an animal.

5. The method of claim 1, wherein said cell is a brain cell, a pancreatic cell, a kidney cell, a cardiac cell, a neuronal cell, or a thyroid cell.

6. A method for inhibiting amyloidosis in a patient comprising administering to said patient an amount effective to inhibit plaque formation of a compound selected from
(S)6-(2-Imidazol-1-yl-1-phenyl-ethoxy)-5-phenethyl-3,4-dihydro-2H-naphthalen-1-one;
6-[2-(1H-Imidazol-4-yl)-ethoxy]-3,4-dihydro-2H-naphthalen-1-one;
E-(+/−)6-(2-Imidazol-1-yl-1-phenyl-ethoxy)-2-thiophen-2-ylmethylene-3,4-dihydro-2H-naphthalen-1-one;
6-[1-(4-Chloro-phenyl)-2-imidazol-1-yl-ethoxy]-3,4-dihydro-2H-napththalen-1-one racemic;
(R)6-(2-Imidazol-1-yl-1-phenyl-ethoxy)-3,4-dihydro-2H-naphthalen-1-one;
6-(2-Imidazol-1-yl-1-phenyl-ethoxy)-4-phenyl-3,4-dihydro-2H-naphthalen-1-one racemic;
6-(2-Imidazol-1-yl-1-phenyl-ethoxy)-5-isopropoxymethyl-3,4-dihydro-2H-naphthalen-1-one;
(S)6-(2-Imidazol-1-yl-1-phenyl-ethoxy)-5-phenylaminomethyl-3,4-dihydro-2H-naphthalen-1-one;
(S)6-(2-Imidazol-1-yl-1-phenyl-ethoxy)-5-[2-(4-fluorophenyl)ethyl]-3,4-dihydro-2H-naphthalen-1-one;
(S)5-Benzenesulfonylmethyl-6-(2-imidazol-1-yl-1-phenyl-ethoxy)-3,4-dihydro-2H-naphthalen-1-one;
(S)6-(2-Imidazol-1-yl-1-phenyl-ethylsulfanyl)-5-phenethyl-3,4-dihydro-2H-naphthalen-1-one;
(S)6-(2-Imidazol-1-yl-1-phenyl-ethoxy)-5-(2-pyridin-2-yl-ethyl)-3,4-dihydro-2H-naphthalen-1-one;
6-(2-Imidazol-1-yl-1-phenyl-ethoxy)-5-(2-pyridin-4-yl-ethyl)-3,4-dihydro-2H-naphthalen-1-one;
4-(5-Oxo-1-phenethyl-5,6,7,8-tetrahydro-naphthalen-2-yloxy)-4-phenyl-butyric acid;
6-[2-(3-Benzyl-3H-imidazol-4-yl)-ethoxy]-5-phenethyl-3,4-dihydro-2H-naphthalen-1-one; trifluoro-acetate;
(S)[1-{(4-Benzyloxy-benzyl)-[(2-methyl-2-phenyl-propylcarbamoyl)-methyl]-carbamoyl}-2-(3H-imidazol-4-yl)-ethyl]-carbamic acid benzyl ester;
(S)[2-(1H-Imidazol-4-yl)-1-((4-methyl-benzyl)-{[(1-phenyl-cyclobutylmethyl)-carbamoyl]-methyl}-carbamoyl)-ethyl]-carbamic acid benzyl ester;
1-Methyl-4-(3-chlorophenyl)-6-[(4-chlorophenyl)-(1-methylimidazol-5-yl)aminomethyl]-2,3-dihydroquinolin-2-one; and
(S)[1-{(4-Benzyloxy-benzyl)-[(2-benzyloxy-ethylcarbamoyl)-methyl]-carbamoyl}-2-(1H-imidazol-4-yl)ethyl]-carbamic acid benzyl ester.

7. The method of claim 6, wherein said amyloidosis is associated with Alzheimer's disease.

8. A method of treating Alzheimer's disease comprising administering to a patient in need of treatment an effective amount of
(S)6-(2-Imidazol-1-yl-1-phenyl-ethoxy)-5-phenethyl-3,4-dihydro-2H-naphthalen-1-one.
